(19) United States
(12) Patent Application Publication
(10) Pub. No.: US 2018/0168952 A1
(43) Pub. Date: Jun. 21, 2018

(54) COMPOSITION AND APPLICATION IN BABY LOTION THEREOF

(71) Applicant: Shanghai Children's Nutrition Center Co., Ltd, Shanghai (CN)

(72) Inventor: WENFENG DENG, Shanghai (CN)

(21) Appl. No.: 15/842,877

(22) Filed: Dec. 15, 2017

(30) Foreign Application Priority Data
Dec. 15, 2016 (CN) 201611162789.6

(51) Int. Cl. A61K 8/14; A61K 8/49; A61K 8/9789; A61K 8/9794; A61Q 19/00; A61K 8/44; A61K 8/37; A61K 8/31; A61K 8/60; A61K 8/34; A61K 8/81

(52) U.S. Cl. CPC A61K 8/14; A61K 8/4946; A61K 8/9789; A61K 8/9794; A61Q 19/00; A61K 8/44; A61K 2800/52; A61K 8/31; A61K 8/604; A61K 8/342; A61K 8/60; A61K 8/8152; A61K 8/37

(57) ABSTRACT

The present disclosure relates to a composition and application in baby lotion thereof. The composition consists of allantoin, grapefruit seed extract and garlic extract; the composition of the present invention effectively moisturizes the skin and reduces redness of skin allergy. Besides, the composition is prepared into liposomes to increase the absorption of actives through unique liposome encapsulation technology, so that the amount of active added is reduced, and the side effects of the product is reduced.

### FIELD OF THE INVENTION

The present invention relates to the field of baby skin care products, more specifically to a composition for alleviating the skin sensitization and redness and application in baby lotion thereof.

### BACKGROUND OF THE INVENTION

Baby has thin skin cuticle, tender skin, more sweat secretion, incomplete skin development and poor ability in controlling acid and base. The skin is only protected from bacterial infection by a layer of acid protective film on the skin surface. If skin is stimulated by the outside world, allergies, irritation and other phenomena occur.

Babies' skin need to be applied with moisturizer with natural moisturizing ingredients due to young and sensitive skin and poor resistance to the environment. There exist a great variety of moisturizers on the market specialized for babies, and these moisturizers usually contain moistures and natural mineral oils which effectively nourishes the baby's skin and prevents chapped skin, moisturizes the skin, but they are oily and not suitable for use in high temperature season and babies with oily skin. Therefore, some moisturizing products containing ingredients such as glycerin and marigolds occur. Especially in the dry autumn and winter seasons, the baby's delicate skin needs more care.

With the improvement of people's living standards, constant renewal of consumer attitudes and the increasing emphasis on environmental issues, moisturizing products containing natural active ingredients attract more and more attention. The natural active ingredients have certain effects and no side effects, which is less harmful to the health of babies and young children.

Chinese patent CN 102440936A discloses a non-irritating moisturizing lotion for baby use, which contains chamomile, aloe vera juice, olive oil essence, lavender, vitamin B5, citric acid, propolis and fresh milk, which can not only clean the skin but also has the effect of anti-bacteria and increasing immunity.

Chinese patent CN 103251531B discloses a soothing propolis cream, which mainly comprises a plurality of pure natural plant extracts of Centella asiatica, Polygonum cuspidatum, Scutellaria baicalensis Georgi, tea leaves, licorice root, maternal chrysanthemum, and rosemary incense, and also contains propolis extract. It has anti-inflammatory and immunomodulatory effects that help to care, repair, nourish and protect delicate baby's skin. However, the results show that the moisturizing product with simple composition is more suitable for babies, which prevents allergic reactions of various components to the baby's skin.

Patent CN 105534850 provides a propolis-containing moisturizing product for babies, which has the effect of moisturizing the skin;

Patent CN 105434193A relates to a camellia oil lotion for baby, which has the effect of moisturizing the skin and preventing dry and chapped skin.

Most existing common lotions on the market use mineral oil as the base oil, which accumulate the grease, block cell metabolism is blocked and accelerate cell senescence if used for long time. At the same time, the effectiveness of baby moisturizing products on the market is not significant, the skin problems, such as allergic irritation cannot be improved significantly. The main reason for this problem is that the active substance has no treatment effect due to small absorption. And if the content of the active ingredient is increased, the side effects of the product are also increased, thereby resulting in other adverse reactions.

### SUMMARY OF THE INVENTION

The purpose of the present invention is to provide a composition and the application thereof in baby lotion. The composition of the present invention effectively moisturizes the skin and relieves the skin sensitization and redness. The composition is prepared into liposomes Through the unique liposome encapsulation technology to increase the absorption of actives, thereby reducing the amount of actives added and reducing the side effects of the product.

The purpose of the present invention is achieved by the following technical solutions.

A composition comprises following ingredients in parts by weight:

| | |
|---|---|
| allantoin | 50-70 parts; |
| grapefruit seed extract | 10-30 parts; and |
| garlic extract | 20-40 parts. |

Further, in the present invention, in order to improve the bioavailability of the active substance, the present invention prepares the composition into a liposome.

A liposome encapsulating the composition is obtained by the following steps:

step a. dissolving the composition in 0.05 mol/L phosphate buffered saline having a pH value of 7 to prepare a 20 mg/ml composition solution; heating to 50-80° C., maintaining a constant temperature;

step b. dissolving lecithin and cholesterol by a mass ratio of 1:1 in anhydrous ethanol to obtain a mixed solution;

step c. adding the mixed solution into the composition solution maintained a constant temperature in step a under stirring, stirring for 30-50 min, rotary evaporating to remove residual ethanol; performing ultrasonic treatment for 5˜10 min to obtain the liposome.

The sonication power is 200 W. The present invention has found that the mixed composition can be mixed uniformly after ultrasonic treatment, the stability of allantoin, grapefruit seed extract and garlic extract in the recipe can be improved, and the period of isolation of bad odors from garlic extract will be prolonged after ultrasonic treatment.

On the other hand, the present invention further provides the application of the composition in baby lotion.

Further, the present invention provides a baby lotion comprising the liposome encapsulating the composition. The baby lotion comprises following ingredients in parts by weight:

| | | |
|---|---|---|
| liposome encapsulating the composition | 0.1-5 | parts; |
| humectant | 0.1-10 | parts; |
| skin conditioner | 0.1-5 | parts; |
| emulsifier | 5-30 | parts; |
| stabilizer | 0.01-1 | part; |
| essence | 0.01-1 | part; and |
| balance being water. | | |

The humectant includes but not limited to glycerinum, sodium polyglutamate and hyaluronic acid.

The skin conditioner includes but not limited to polyacrylate-13, polyisobutene, polysorbate 20.

The emulsifier includes but not limited to isopropyl palmitate, liquid paraffin, cetyl alcohol, beewax, glycol palmitate, C14-22 alcohol, and C12-20 alkyl polyglycoside.

The stabilizer includes but not limited to phenoxyethanol, ethylhexylglycerol

The essence includes but not limited to natural orange oil, natural jasmine oil.

The advantages and benefits of the present invention over the prior art.

(1) The composition for alleviating skin hypersensitivity and redness of the present invention consists of allantoin, grapefruit seed extract and garlic extract. Allantoin keeps the moisture of skin, moisturizes the skin, prevents rahagades, acne and chilblain; grapefruit seed extract has antibacterial and anti-inflammatory effects; garlic extract has the effect of enhancing plasminogen activity and firming the skin. And the effect of mixing these three ingredients is obviously better than that of the single component.

(2) The invention prepares the composition for alleviating skin hypersensitivity and redness into a liposome and adds the liposome into the product to improve the stability of the garlic extract and isolate the bad smell of the garlic extract; on the other hand, the preparation of the composition into liposomes increases the absorption of actives to reduce the amount of actives added to reduce the side effects of the product.

(3) After ultrasonic treatment, the stability of the allantoin, grapefruit seed extract and garlic extract in the formulation is increased, and the time for isolating the bad smell of the garlic extract is prolonged.

### DETAILED DESCRIPTION OF THE INVENTION

According to the disclosure and teachings of the foregoing specification, those skilled in the art may also make changes and modifications to the above-described embodiments. Therefore, the present invention is not limited to the specific embodiments disclosed and described above, and some modifications and alterations to the present invention should also fall within the protection scope of the claims of the present invention. In addition, although some specific terms are used in this specification, these terms are merely for convenience of description and do not limit the present invention in any way.

### Embodiments 1-3

Specific formulations of the composition provided by the present invention are as follows. The embodiments are not intended to limit the present invention. Unless otherwise specified, the amounts of active ingredients are parts by weight.

| | Embodiment 1 | Embodiment 2 | Embodiment 3 |
|---|---|---|---|
| Allantoin | 50 | 60 | 70 |
| Grapefruit seed extract | 15 | 20 | 10 |
| Garlic extract | 35 | 20 | 20 |

A liposome encapsulating the composition is obtained by the following steps:

step a. dissolving the composition in 0.05 mol/L phosphate buffered saline having a pH value of 7 to prepare a composition solution of 20 mg/ml; heating to 50° C., maintaining a constant temperature;

step b. dissolving lecithin and cholesterol by a mass ratio of 1:1 in anhydrous ethanol to obtain a mixed solution;

step c. adding the mixed solution into the composition solution under a constant temperature in the step a under stirring, stirring for 30 min, rotary evaporating to remove residual ethanol; and performing ultrasonic treatment for 10 min under 200 W.

### Embodiments 4-8

The embodiments of liposomes application in baby lotions according to the present invention are as follows. The total amount of the components A, B and C is 100 parts:

| | | Embodiment | Embodiment | Embodiment | Embodiment | Embodiment |
|---|---|---|---|---|---|---|
| | | 4 | 5 | 6 | 7 | 8 |
| A | Liposomes of the | 2 | 0 | 0 | 0 | 0 |
| phase | composition in | | | | | |
| | embodiment 1 | | | | | |
| | (drug loading | | | | | |
| | capacity is 50%) | | | | | |
| | Allantoin | 0 | 0.5 | 0.5 | 0 | 0 |
| | Grapefruit seed | 0 | 0.15 | 0 | 0 | 0 |
| | extract | | | | | |
| | Garlic extract | 0 | 0.35 | 0 | 0.35 | 0 |
| | Sodium | 0.5 | 0.5 | 0.5 | 0.5 | 0.5 |
| | L-glutamate | | | | | |
| | Water | margin | margin | margin | margin | margin |
| B | Isopropyl | 8 | 8 | 8 | 8 | 8 |
| phase | palmitate | | | | | |
| | Liquid paraffin | 8 | 8 | 8 | 8 | 8 |
| | C12-20 alkyl | 2 | 2 | 2 | 2 | 2 |
| | polyglycoside | | | | | |
| | Cetyl alcohol | 1 | 1 | 1 | 1 | 1 |
| | Hydroxyethyl | 1.2 | 1.2 | 1.2 | 1.2 | 1.2 |
| | palmitate | | | | | |
| | Phenoxyethanol | 0.3 | 0.3 | 0.3 | 0.3 | 0.3 |
| C | Polysorbate 20 | 0.2 | 0.2 | 0.2 | 0.2 | 0.2 |
| phase | Polyacrylate-13 | 0.2 | 0.2 | 0.2 | 0.2 | 0.2 |
| | Natural essence | 0.1 | 0.1 | 0.1 | 0.1 | 0.1 |

The preparation method of the baby lotion is as follows: adding the ingredients in A phase into water in sequence, and stirring for 40 min at room temperature; heating the B phase to 85° C., and stirring to dissolve the B phase; adding the B phase to the A phase, stirring and emulsifying homogenously for 30 min. Adding the C phase into the A phase, stirring for 30 min at 50° C.

The products obtained in the above embodiment 4 and embodiment 5 were placed in normal temperature and 45° C. respectively to test the stability of the products and the change of the active substance content. The results are shown in the following table.

| Allantoin | | Grapefruit seed extract | | Garlic extract | |
|---|---|---|---|---|---|
| Normal | | Normal | | Normal | |
| temperature | 45° C. | temperature | 45° C. | temperature | 45° C. |

Test Results of The Active Substance Content in Embodiment 4

| | | | | | | |
|---|---|---|---|---|---|---|
| Start | 0.5 | 0.49 | 0.15 | 0.15 | 0.35 | 0.35 |
| 1 month | 0.48 | 0.48 | 0.14 | 0.14 | 0.33 | 0.32 |
| 2 months | 0.48 | 0.47 | 0.14 | 0.13 | 0.32 | 0.30 |
| 3 months | 0.47 | 0.47 | 0.13 | 0.13 | 0.3 | 0.28 |

Test Results of The Active Substance Content in Embodiment 5

| | | | | | | |
|---|---|---|---|---|---|---|
| Start | 0.5 | 0.49 | 0.15 | 0.15 | 0.35 | 0.35 |
| 1 month | 0.48 | 0.47 | 0.13 | 0.13 | 0.32 | 0.30 |
| 2 months | 0.47 | 0.46 | 0.11 | 0.10 | 0.30 | 0.26 |
| 3 months | 0.46 | 0.45 | 0.10 | 0.08 | 0.25 | 0.21 |

| | Embodiment 4 | | Embodiment 5 | |
|---|---|---|---|---|
| | Normal | | Normal | |
| | temperature | 45° C. | temperature | 45° C. |
| Start | Normal | Normal | Normal color; | Normal color; |
| | | | garlic smell | garlic smell |
| 1 month | Normal | Normal | Normal color; | Slightly yellow; |
| | | | garlic smell | garlic smell |
| 2 months | Normal | Slightly yellow; | Slightly yellow; | Slightly yellow; |
| | | normal smell | garlic smell | garlic smell |
| 3 months | Normal | Slightly yellow; | Slightly yellow; | Slightly yellow; |
| | | normal smell | normal smell | obvious garlic smell |

The above test results can be seen that the composition encapsulated by liposomes technology improves the stability of the grapefruit seed extract and garlic extract components in the formulation. And the composition encapsulated by liposomes is conducive to cover the special smell of the extract and enhance the comfort of consumers.

In order to verify the effect of the prepared diaper cream, sixty one-year-old babies were randomly selected, and randomly divided into 3 groups of 20 people to test in October. The divided three groups were respectively applied with lotions of embodiment 4, embodiment 5, and embodiment 6 each time in the morning and evening; after two weeks, the performance and results of the lotions for each group of testers were shown in the table below.

| | Embodiment 4 | Embodiment 5 | Embodiment 6 |
|---|---|---|---|
| Humidity | Strong | Strong | Medium |
| Moisture durability | Durable | Common | Common |
| Spread dispersion | Good | Good | Good |
| Skin irritation | No stimulation | No stimulation | No stimulation |
| Recovery rate of dry | 95% | 78% | 64% |
| skin | | | |
| Recovery rate of | 83% | 67% | 48% |
| skin redness | | | |

It can be seen from the above results that moisturizing effect of lotion with liposomes is good and durable. It has high recovery efficiency of dry and chapped skin and skin redness, and the effect is better than only adding the composition or not adding the composition. Therefore, the composition is prepared into liposomes to increase the absorption of the active material and reduce the amount of the active material added and the side effects of the product.

Forty babies aged 1 were selected randomly, divided into 2 groups, and tested in October. The two groups are respectively applied with lotions of embodiment 6 and embodiment 7 for one time in every morning and every evening; after two weeks, the performance and results of the lotion for each group of testers were shown in the table below.

| | Embodiment 6 | Embodiment 7 | Embodiment 8 |
|---|---|---|---|
| Humidity | Medium | Medium | Medium |
| Moisture durability | Common | Common | Common |
| Spread dispersion | Good | Good | Good |
| Skin irritation | No Stimulation | No Stimulation | No Stimulation |
| Recovery rate of dry | 65% | 69% | 64% |
| skin | | | |
| Recovery rate of skin | 58% | 55% | 48% |
| redness | | | |

It can be seen from the above results that the moisturizing effect, durability, recovery rate of dry skin and recovery rate of skin redness of the lotion added with a certain kind of extract is not better than the lotion added with the liposomes encapsulating the composition in embodiment 4. Therefore, mixing three ingredients and encapsulating active ingredients are beneficial to improve the effect of product.

### Embodiment 9

Verification the Effect of Ultrasonic Treatment

The liposomes of the composition of embodiment 3 is obtained according to the following steps:

step a. dissolving the composition in 0.05 mol/L phosphate buffered saline having a pH value of 7 to prepare a composition solution of 20 mg/ml solution; heating to 50° C., maintaining a constant temperature;

step b. dissolving lecithin and cholesterol by a mass ratio of 1:1 in anhydrous ethanol to obtain a mixed solution;

step c. adding the mixed solution into the composition solution maintained a constant temperature in step a under stirring, stirring for 30 min, rotary evaporating to remove residual ethanol; performing ultrasonic treatment for 10 min, and obtaining the liposomes encapsulating the composition, and the ultrasonic treatment is performed under 200 W.

The liposomes of the composition of embodiment 3 is obtained according to the following steps as a contrast:

step a. dissolving the composition in 0.05 mol/L phosphate buffered saline having a pH value of 7 to prepare a composition solution of 20 mg/ml; heating to 50° C., maintaining a constant temperature;

step b. dissolving lecithin and cholesterol by a mass ratio of 1:1 in anhydrous ethanol to obtain a mixed solution;

step c. adding the mixed solution into the composition solution maintained a constant temperature in step a under stirring, stirring for 30 min, rotary evaporating to remove residual ethanol; and obtaining the liposomes encapsulating the composition.

The lotion is prepared based on the following formulation (in parts by weight, 100 parts in total): adding the ingredients in A phase into water in sequence, and stirring for 40 min at room temperature; heating the B phase to 85° C., and stirring to dissolve the B phase; adding the B phase to the A phase, stirring and emulsifying homogenously for 30 min. Adding the C phase into the A phase, stirring for 30 min at 50° C.

| | Embodiment 9 | | Contrast | |
|---|---|---|---|---|
| A | Liposomes of the | 2 | Liposomes of the | 2 |
| phase | composition of | | contrast | |
| | embodiment 3 | | | |
| | Allantoin | 0 | Allantoin | 0 |
| | Grapefruit seed | 0 | Grapefruit seed | 0 |
| | extract | | extract | |
| | Garlic extract | 0 | Garlic extract | 0 |
| | Sodium | 10 | Sodium | 10 |
| | l-glutamate | | l-glutamate | |
| | Water | Balance | Water | Balance |
| B | Isopropyl | 15 | Isopropyl | 15 |
| phase | palmitate | | palmitate | |
| | Liquid paraffin | 10 | Liquid paraffin | 10 |
| | C12-20 alkyl | 2 | C12-20 alkyl | 2 |
| | polyglycoside | | polyglycoside | |
| | Cetyl alcohol | 1 | Cetyl alcohol | 1 |
| | Hydroxyethyl | 2 | | 2 |
| | palmitate | | | |
| | Pphenoxyethanol | 1 | pPhenoxyethanol | 1 |
| C | Phenoxyethanol | 2 | | 2 |
| phase | Polysorbate 20 | 3 | | 3 |
| | Polyacrylate-13 | 1 | | 1 |

| Allantoin | | Grapefruit seed extract | | Garlic extract | |
|---|---|---|---|---|---|
| Normal | | Normal | | Normal | |
| temperature | 45° C. | temperature | 45° C. | temperature | 45° C. |

Test Results of The Content of The Actives in Embodiment 9

| | | | | | | |
|---|---|---|---|---|---|---|
| Start | 0.7 | 0.7 | 0.1 | 0.1 | 0.2 | 0.2 |
| 1 month | 0.7 | 0.7 | 0.1 | 0.1 | 0.18 | 0.17 |
| 2 months | 0.7 | 0.69 | 0.1 | 0.09 | 0.16 | 0.15 |
| 3 months | 0.69 | 0.68 | 0.09 | 0.08 | 0.15 | 0.14 |

Test Results of The Content of The Actives in Contrast

| | | | | | | |
|---|---|---|---|---|---|---|
| Start | 0.7 | 0.7 | 0.1 | 0.1 | 0.2 | 0.2 |
| 1 month | 0.68 | 0.66 | 0.09 | 0.08 | 0.17 | 0.15 |
| 2 months | 0.67 | 0.65 | 0.08 | 0.07 | 0.14 | 0.11 |
| 3 months | 0.66 | 0.64 | 0.07 | 0.06 | 0.11 | 0.06 |

It can be seen from the above testing result that the liposomes encapsulating the composition after ultrasonic treatment improves the stability of the grapefruit seed extract and garlic extract in the formulation, the releasing time of bad smell of garlic is started from 24 months to 36 months.

According to the disclosure and teachings of the foregoing specification, those skilled in the art may also make changes and modifications to the above-described embodiments. Therefore, the present invention is not limited to the specific embodiments disclosed and described above, and some modifications and alterations to the present invention should also fall within the protection scope of the claims of the present invention. In addition, although some specific terms are used in this specification, these terms are merely for convenience of description and do not limit the present invention in any way.

What is claimed is:

1. A composition, comprising following ingredients in parts by weight:

| | |
|---|---|
| allantoin | 50-70 parts; |
| grapefruit seed extract | 10-30 parts; and |
| garlic extract | 20-40 parts. |

2. A method for preparing a liposome encapsulating the composition of claim 1, wherein the method comprises following steps:
step a. dissolving the composition of claim 1 in 0.05 mol/L phosphate buffered saline having a pH value of 7 to prepare a composition solution of 20 mg/ml; heating to 50-80° C. and maintaining a constant temperature;
step b. dissolving lecithin and cholesterol by a mass ratio of 1:1 in anhydrous ethanol to obtain a mixed solution;
step c. adding the mixed solution into the composition solution obtained in the step a under stirring, stirring for 30-50 min, rotary evaporating to remove residual ethanol; performing ultrasonic treatment for 5˜10 min to obtain the liposome.

3. The method for preparing a liposome of claim 2, wherein, ultrasonic treatment is performed under 200 W.

4. A baby lotion comprising the liposome of claim 2, wherein the baby lotion comprises following ingredients in parts by weight:

| | | |
|---|---|---|
| the liposome | 0.1-5 | parts; |
| humectant | 0.1-10 | parts; |
| skin conditioner | 0.1-5 | parts; |
| emulsifier | 5-30 | parts; |
| stabilizer | 0.01-1 | part; |
| essence | 0.01-1 | part; and |
| balance being water. | | |

5. The baby lotion of claim 4, wherein, the humectant is one or more of glycerinum, sodium polyglutamate and hyaluronic acid.

6. The baby lotion of claim 4, wherein, the skin conditioner is one or more of polyacrylate-13, polyisobutene and polysorbate 20.

7. The baby lotion of claim 4, wherein, the emulsifier is one or more of isopropyl palmitate, liquid paraffin, cetyl alcohol, beewax, glycol palmitate, C14-22 alcohol, and C12-20 alkyl polyglycoside.

8. The baby lotion of claim 4, wherein, the stabilizer is one or more of phenoxyethanol and ethylhexylglycerin.
